(19) United States
(12) Patent Application Publication
(10) Pub. No.: US 2020/0315491 A1
(43) Pub. Date: Oct. 8, 2020

(54) METHOD AND APPARATUS FOR CONTROLLING THE GENERATION OF A MAGNETIC RESONANCE IMAGING SEQUENCE

(71) Applicants: Siemens Healthcare Limited, Camberley (GB); King's College London, London (GB); Siemens Healthcare GmbH, Erlangen (DE)

(72) Inventors: Ronald Mooiweer, London (GB); Rainer Schneider, Erlangen (DE); Radhouene Neji, Camberley (GB); Reza Razavi, London (GB); Tobias Schaeffter, London (GB); Sebastien Roujol, London (GB)

(73) Assignees: Siemens Healthcare Limited, Camberley (GB); King's College London, London (GB); Siemens Healthcare GmbH, Erlangen (DE)

(21) Appl. No.: 16/839,227

(22) Filed: Apr. 3, 2020

(30) Foreign Application Priority Data
Apr. 3, 2019 (GB) 1904699.4

(51) Int. Cl. A61B 5/055; G01R 33/28; G01R 33/567; G01R 33/58; G01R 33/48; A61B 5/01; A61B 5/00

(52) U.S. Cl. CPC A61B 5/055; G01R 33/287; G01R 33/5673; G01R 33/58; A61B 2505/05; A61B 5/015; A61B 5/7285; A61B 5/6852; A61B 2560/0223; G01R 33/4804

(57) ABSTRACT

A magnetic resonance (MR) apparatus and method for controlling a generation of an imaging sequence for imaging a subject. The method includes generating an MR tracking sequence for tracking a position of an MR active device located in the subject; obtaining MR signals detected by the MR active device as a result of the generated tracking sequence; processing the obtained MR signals to determine the position of the MR active device; determining whether a trigger condition is satisfied by comparing the determined position of the MR active device to a predetermined trigger position; and generating the imaging sequence if the trigger condition is satisfied, wherein if the trigger condition is not satisfied, the imaging sequence is not generated.

### TECHNICAL FIELD

The present disclosure is directed towards a method and apparatus for controlling the generation of a magnetic resonance (MR) imaging sequence, and in particular is directed towards triggering the generation of the imaging sequence for imaging a subject based on a detected motion level of the subject.

### BACKGROUND

Magnetic resonance, MR imaging may be used to provide guidance in catheter-based interventions. MR imaging provides benefits compared to existing approaches such as X-ray fluoroscopy at least because MR imaging does not involve generating ionizing radiation.

Two different approaches are commonly used to track the catheter using MR imaging: passive tracking and active tracking.

MR-guided passive tracking is for visualizing a device within MR images based on the negative or positive contrast generated by intrinsic material characteristics of the device (e.g. its magnetic susceptibility). The contrast can be created and enhanced by incorporating ferromagnetic or paramagnetic materials into the device, or by using contrast agents. Specific imaging sequences have also been proposed to improve the visualization.

MR guided active tracking uses MR active devices with receive coils, antennas, or other sensors, to generate signals for localization. The present disclosure is focused on MR guided active tracking.

When performing MR guided active tracking, it may be desirable to trigger the generation of an imaging sequence. For example, it may be desirable to trigger the generation of an MR thermometry sequence for imaging the temperature of a region of interest of the subject. One particular application of MR thermometry is in MR guided cardiac ablations of cardiac arrhythmias in which the MR apparatus tracks the cardiac ablation catheter. MR thermometry can be used during a cardiac ablation procedure so as to provide a real-time assessment of the ablation lesions.

It is desirable to trigger the generation of the MR thermometry sequence such that it occurs during a particular stage of the cardiac cycle (heartbeat). One reason for this is that it helps ensure that each MR imaging sequence, generated across a plurality of cycles, occurs during the same stage of the cardiac cycle. Another benefit is that the MR imaging sequence can be triggered to occur during a particular quiescent stage of the cardiac cycle such that the effect of motion on the MR imaging sequence is reduced.

A particular existing approach of triggering the MR imaging sequence comprises using an electrocardiography, ECG, apparatus to monitor the electrical activity during the cardiac cycle. The ECG information is used to trigger the generation of the MR imaging sequence such that the MR imaging sequence occurs during a particular stage of the cardiac cycle. That is, the existing approach uses ECG-triggered image acquisitions.

A problem with this existing approach is that the ECG-triggering can be unreliable. One reason for this is due to the influence of RF equipment and magnetic field gradients generated by the MR apparatus. Another reason for this is because of the additional RF equipment in the form of the ablation device which decreases the reliability of the ECG-triggering. In addition, MR thermometry sequences and other sequences which use echo-planar imaging techniques also decrease the reliability of ECG-triggering because these imaging techniques are associated with strong magnetic field gradients.

It is an object of the present disclosure to provide an improved approach for triggering the generation of imaging sequences, and in particular an approach for triggering the generation of imaging sequences that is not affected by or is less affected by RF equipment and magnetic field gradients than the existing ECG-triggering based approach.

### Summary

According to a first aspect of the present disclosure, there is performed a method performed by a magnetic resonance, MR, apparatus for controlling the generation an imaging sequence for imaging a subject. The method comprises:

generating an MR tracking sequence for tracking the position of an MR active device located in the subject;

obtaining MR signals detected by the MR active device as a result of the generated tracking sequence;

processing the obtained MR signals to determine the position of the MR active device;

determining whether a trigger condition is satisfied by comparing the determined position of the MR active device to a predetermined trigger position; and

generating the imaging sequence if the trigger condition is satisfied, wherein if the trigger condition is not satisfied, the imaging sequence is not generated.

Significantly, the present disclosure measures the position of the MR active device located in the subject using an MR tracking sequence, and uses the position of the MR active device to determine when to generate the imaging sequence. In this way, the present disclosure triggers the imaging sequence based on the detected position of the MR active device in the subject. It will be appreciated that for an otherwise stationary MR active device located in the subject, the MR active device may be moved due to subject motion, and especially regular, repeatable, subject motion such as cardiac motion and/or respiratory motion. This regular, repeatable, subject motion may be considered as periodic motion.

For example, the MR active device may be located within the cardiac region of the subject and may move as a result of cardiac motion during the cardiac cycle. Because of this the position of the MR active device corresponds to the current state of the cardiac cycle. The present disclosure sets a predetermined trigger position of the MR active device and uses this predetermined trigger position to determine whether to generate the imaging sequence. It will be appreciated that the predetermined trigger position could correspond to a particular state of the cardiac cycle, respiratory cycle or other involuntary subject motion cycle depending on the location of the MR active device in the subject. By selecting a desired predetermined trigger position the present disclosure is able to control when the imaging sequence is generated during the motion cycle.

In this way, the present disclosure provides an approach for controlling the generation of imaging sequences which does not require the use of an ECG apparatus. Instead, the present disclosure tracks the position of the MR active device and uses the position to trigger the generation of the imaging sequence. The present disclosure is therefore not affected by or at least less affected by RF equipment and magnetic field gradients than the existing ECG-triggering based approach. The predetermined trigger position may be selected such that the imaging sequence is generated during a quiescent phase of a motion cycle of the subject, such as a quiescent phase of the cardiac cycle and/or a quiescent phase of the respiratory cycle. The present disclosure also makes use of the existing MR active device used in active tracking and thus requires no additional equipment.

The present disclosure is also beneficial over existing passive approaches of triggering imaging sequences such as the use of image navigators. Image navigators require the generation of images of sufficient detail to allow for the subject motion such as cardiac motion to be modelled. This means that each image navigator sequence needs a relatively long acquisition time of approximately 50 ms. Additional time is required to reconstruct the image data from the obtained data in the frequency domain and process the images to estimate the subject motion. The present disclosure overcomes these problems by generating an MR tracking sequence for tracking the position of the MR active device rather than imaging the subject itself to assess the subject motion. The MR tracking sequences can be simple and do not need a long acquisition time. The position of the MR active can be obtained in the frequency domain and, optionally, converted into a spatial coordinate. Reconstruction of the MR image from the k-space data is not required. In this way, the MR tracking sequences and the subsequent processing operations in accordance with the present disclosure can be performed in real-time.

The imaging sequence may be for imaging a cardiac region of the subject. Step (a) may comprise generating an MR tracking sequence for tracking the position of the MR active device located in the cardiac region of the subject. In this example, the MR active device is located in the cardiac region of the subject and is subject to regular repeatable motion in the form of at least cardiac motion due to the cardiac cycles of the subject. The MR active device may also be subject to other subject motion such as respiratory motion if the method is performed during a free-breathing MR imaging procedure.

The present disclosure is not limited to cardiac imaging, and is not limited to tracking the position of the MR active device located in the cardiac region. The imaging of other regions of the subject is within the scope of the present disclosure. For example, the present disclosure can be performed with and achieve benefits for any region of the subject where an MR active device may be introduced and which may be subject to motion such as regular, repeatable, subject motion which can be used to trigger the generation of imaging sequences.

For example, the present disclosure may be used to trigger the generation of imaging sequences based on respiratory motion of the subject during a tumour ablation procedure in the abdomen, kidney or pancreas of the subject. In these examples, the imaging sequence could be triggered based on the breathing phase, e.g. so that the imaging sequence is generated during a quiescent phase of the respiratory cycle. In these example, the imaging sequence may be for imaging the tumour in the abdomen, kidney, or pancreas of the subject. Step (a) of the method may comprise generating an MR tracking sequence for tracking the position of the MR active device located in the abdomen, kidney or pancreas of the subject.

Particular benefits are achieved by the present disclosure in relation to tracking cardiac motion and triggering imaging sequences based on cardiac motion at least because the approach of the present disclosure overcomes the problems in relation to ECG-apparatuses as mentioned above.

If the trigger condition is not satisfied, the method may comprise repeating steps (a) to (e). If the trigger condition is satisfied, the method may comprise repeating steps (a) to (e) until the trigger condition is satisfied. This means that the MR tracking sequences may be continuously generated until the trigger condition is satisfied.

Generating the imaging sequence may comprise generating the imaging sequence a predetermined time after the trigger condition is satisfied. The predetermined time may be selected such that the imaging sequence is generated during a specified time point in the motion cycle such as the cardiac cycle of the subject. The specified time point may correspond to a quiescent phase of the motion cycle. In this way, the generation of the imaging sequence is delayed after the trigger condition is satisfied until the quiescent phase. It will be appreciated that the amount of delay will depend on the particular predetermined trigger position and/or the trigger condition.

During the delay period before the imaging sequence is generated, the method may further comprise generating a plurality of tracking sequences for tracking the position of the MR active device located in the subject. Signals obtained from these tracking sequences may be used improve the filtering of the determined MR active device positions until the imaging sequence is generated. Signals obtained from these tracking sequences may be used to determine if the position of the MR active device prior to the generation of the imaging sequence corresponds to a quiescent phase of the motion cycle of the subject. This may comprise comparing the determined position of the MR active device to another predetermined trigger position that represents the quiescent phase of the motion cycle. The method may comprise not generating the imaging sequence and repeating steps (a) to (e) if it is determined that the position of the MR active device prior to the generation of the imaging sequence does not correspond to the quiescent phase of the motion cycle.

Immediately prior to generating the imaging sequence, the method may comprise: generating an MR tracking sequence for tracking the position of the MR active device; obtaining MR signals detected by the MR active device as a result of the generated tracking sequence; and processing the obtained MR signals to determine the position of the MR active device.

Here, “immediately prior” will be understood as referring to a short time period before the generation of the imaging sequence. That is, a tracking sequence is generated just before the imaging sequence. The time at which the tracking sequence is generated prior to the generation of the imaging sequence will depend on factors such as the duration of the tracking sequence. For example, if the tracking sequence has a duration of 25 ms then the tracking sequence may be generated between 25 ms and 100 ms, optionally 25 and 50 ms, before the generation of the imaging sequence. Generally, generating the tracking sequence immediately prior to the imaging sequence may mean that the tracking sequence is generated between 1x (the duration of the tracking sequence) and 4x(the duration of the tracking sequence) before the generation of the imaging sequence.

The method may comprise using the determined position of the MR active device to correct the position of an MR image obtained from the imaging sequence. Optionally the MR image is aligned with the position of the MR active device. In this way, a final MR tracking sequence is generated prior to the generation of the imaging sequence so as to reposition the imaging slice.

The method may comprise using the determined position of the MR active device to determine if the position of the MR active device immediately prior to the generation of the imaging sequence corresponds to a quiescent phase of the motion cycle of the subject. This may comprise comparing the determined position of the MR active device to another predetermined trigger position that represents the quiescent phase of the motion cycle. The method may comprise not generating the imaging sequence and repeating steps (a) to (e) if it is determined that the position of the MR active device immediately prior to the generation of the imaging sequence does not correspond to the quiescent phase of the motion cycle. This may be due to a change in the motion cycle of the subject such as due to a change in heart rate, respiratory rate, or arrhythmia. Advantageously, this means that immediately prior to the generation of the imaging sequence, an additional check is performed to determine whether the subject motion cycle is within the quiescent phase and if not, stops the generation of the imaging sequence, and repeats the process of tracking the subject motion until the trigger condition is satisfied.

The imaging sequence may be an MR thermometry sequence. The MR thermometry sequence may be used to provide a real-time assessment of ablation lesions during an MR guided ablation of cardiac arrhythmias. The present disclosure is not limited to MR thermometry sequences and instead any imaging sequences for imaging the subject and which are desired to be triggered to occur during a motion phase. For example, any form of imaging sequence such as any form of echo-planar imaging sequence may be used.

Processing the obtained MR signals to determine the position of the MR active device may comprise processing the MR signals in the frequency domain so as to identify one or more signal peaks in the MR signals. The identified one or more signal peaks in the MR signals correspond to the position of the MR active device in one or more spatial directions. In this way, the position of the MR active device can be determined from the obtained MR signals.

Determining whether the trigger condition is satisfied by comparing the position of the MR active device to the predetermined trigger position may comprise determining whether the position of the MR active device corresponds to the predetermined trigger position. In this way, the imaging sequence may be generated if the position of the MR active device corresponds to the predetermined trigger position.

Determining whether the trigger condition is satisfied by comparing the position of the MR active device to the predetermined trigger position may comprise determining whether the position of the MR active device has exceeded the predetermined trigger position. In this way, the imaging sequence may be generated if the position of the MR active device has exceeded the predetermined trigger position. The trigger condition may be determined to be satisfied if the position of the MR active device corresponds to and has exceeded the predetermined trigger position.

Determining whether the trigger condition is satisfied may comprise determining whether the MR active device is moving in a certain direction and the trigger condition may only be satisfied if the MR active device is moving in the direction. The direction of motion of the MR active device may be determined by comparing the position of the MR active device to previously obtained positions of the MR active device. For example, if the difference between the current position and the previously obtained position has a positive value then the imaging sequence may be generated. If the difference has a negative value, then the imaging sequence may not be generated. This corresponds to determining whether the local gradient is positive or negative. In this way, the imaging sequence may only be generated when the MR active device is moving in a certain direction and may not be generated when the MR active device is moving in the other direction.

Prior to performing steps (a) to (e), the method may comprise performing a calibration phase to determine the predetermined trigger position. Performing the calibration phase may comprise: generating a plurality of the MR tracking sequences over time for tracking the position of the MR active device located in the subject; obtaining MR signals detected by the MR active device as a result of the generated tracking sequences over time; processing the obtained MR signals to determine how the position of the MR active device changes over time; and using the information about how the position of the MR active device changes over time to set the predetermined trigger position. Advantageously, the calibration phase is used to set a particular predetermined trigger position for the subject based on the measured motion of the MR active device during the calibration phase. The motion of the MR active device reflects the motion of the subject.

It will be appreciated that any particular position of the MR active device may be set as the predetermined trigger position. Using the information about how the position of the MR active device changes over time to set the predetermined trigger position may comprise setting an average position of the MR active device as the predetermined trigger position. Using the information about how the position of the MR active device changes over time to set the predetermined trigger position may comprise setting a minimum or maximum position of the MR active device as the predetermined trigger position.

When the predetermined trigger position is a maximum or minimum position of the MR active device, determining whether the position of the MR active device corresponds to a predetermined trigger position may comprise determining whether the position of the MR active device corresponds to global maximum or minimum. The method may comprise disregarding determined local maximums or minimums by considering whether the determined position lies within the threshold of the maximum or minimum detected position of the MR active device. For example, the method may determine whether the determined position lies within the top 25% of determined positions of the MR active device so as to determine whether the position corresponds to a global maximum.

The MR tracking sequence may comprise a spatially non-selective or minimally spatially selective excitation pulse followed by a magnetic field gradient pulse along a first spatial direction. Obtaining the MR signals detected by the MR active device as a result of the generated tracking sequence may comprise obtaining first MR signals detected as a result of the magnetic field gradient pulse along the first spatial direction. The location, in the frequency domain, of the signal peak for the first MR signals corresponds to the position of the MR active device in the first spatial direction.

The MR tracking sequence may further comprise a spatially non-selective or minimally spatially selective excitation pulse followed by a magnetic field gradient pulse along a second spatial direction perpendicular to the first spatial direction Obtaining the MR signals detected by the MR active device as a result of the generated tracking sequence may further comprise obtaining second MR signals detected as a result of the magnetic field gradient pulse along the second spatial direction. The location, in the frequency domain, of the signal peak for the second MR signals corresponds to the position of the MR active device in the second spatial direction.

The MR tracking sequence may further comprise a spatially non-selective or minimally spatially selective excitation pulse followed by a magnetic field gradient pulse along a third spatial direction perpendicular to the first spatial direction and second spatial direction. Obtaining the MR signals detected by the MR active device as a result of the generated tracking sequence may further comprise obtaining third MR signals detected as a result of the magnetic field gradient pulse along the third spatial direction. The location, in the frequency domain, of the signal peak for the third MR signals corresponds to the position of the MR active device in the third spatial direction.

The method may further comprise applying a filter to a time series of determined positions of the MR active device so as to remove unwanted components. The time series of determined positions of the MR active device comprises the position of the MR active determined as a result of the tracking sequence in step (a) and positions of the MR active device determined using at least one previous tracking sequence. That is, the positions determined as a result of the current tracking sequence and a previous N tracking sequences, where N is 1 or more, are filtered so as to remove unwanted components. The unwanted components of the signal may comprise components which are caused by respiratory motion or drift. The method may comprise applying a filter to separate out the unwanted components from the wanted components of the signal based on the different frequencies of the different motion components. For example, the cardiac cycle can be expected to have a frequency of roughly 1 Hz while the respiratory cycle of the subject has a lower frequency of around 0.3 Hz, and motion due to drift will have an even lower frequency. If cardiac motion forms the wanted motion component and respiratory motion and drift are unwanted motion components, then the filter can be used to remove the low frequency components such that only motion due to the cardiac cycle remains. The filter may be a bandpass filter. It will be appreciated that the filter is not required in all embodiments. In particular, the triggering of the imaging sequence may be performed during a breathhold of the subject which will remove the effect of respiratory motion. In addition, the calibration phase described above may be repeated at different times during the tracking of the MR active device so as to adjust the predetermined trigger position so as to compensate for the effect of drift.

The MR active device may be any device suitable for detecting MR signals and providing MR signals to the MR apparatus so that the signals may be used to determine the position of the MR active device. That is, any MR active device suitable for use in MR active tracking may be used. The MR active device may be a receive coil, an antenna or another sensor. The MR active device may comprise a plurality of receive coils and/or a plurality of antennas and/or a plurality of sensors. The MR active device may comprise a combination of one or more of receive coils, antennas, and sensors. In particular preferred examples, the MR active device is a receive coil or comprises at least one receive coil which may in particular be a microcoil.

The MR active device may be associated with or part of an invasive device. The invasive device may be a guide wire, a catheter, an endoscope, a laparoscope or a biopsy needle. The MR active device may be embedded in the invasive device. The invasive device may be associated with a plurality of MR active devices such as a plurality of receive coils. Some or all of the MR active devices may be tracked using the approaches of the present disclosure.

According to a second aspect of the present disclosure, there is provided a magnetic resonance, MR, apparatus. The MR apparatus comprises a gradient arrangement configured to apply a magnetic field gradient; a transmitter configured to apply an excitation pulse to the subject; and a controller in communication with the transmitter, and the gradient arrangement for controlling these components. The controller is configured to perform the following:

control the transmitter and gradient arrangement to generate an MR tracking sequence for tracking the position of an MR active device located in a subject;

obtain MR signals detected by the MR active device as a result of the generated tracking sequence;

process the obtained MR signals to determine the position of the MR active device;

determine whether a trigger condition is satisfied by comparing the determined position of the MR active device to a predetermined trigger position; and

control the transmitter and gradient arrangement to generate an imaging sequence for imaging the subject if the trigger condition is satisfied, wherein if the trigger condition is not satisfied, the imaging sequence is not generated.

If the trigger condition is not satisfied, the controller may be configured to repeat the performance of (a) to (e). If the trigger condition is not satisfied, the controller may be configured to repeat the performance of (a) to (e) until the trigger condition is satisfied.

The controller may be configured to control the transmitter and gradient arrangement to generate the imaging sequence a predetermined time after the trigger condition is determined to be satisfied. The predetermined time may be selected such that the imaging sequence is generated during a specified time point in the motion cycle of the subject. The specified time point may correspond to a quiescent phase of the motion cycle.

Immediately prior to generating the imaging sequence, the controller may be configured to: control the transmitter and gradient arrangement to generate an MR tracking sequence for tracking the position of the MR active device; obtain MR signals detected by the MR active device as a result of the generated tracking sequence; process the obtained MR signals to determine the position of the MR active device; and use the determined position of the MR active device to correct the position of an MR image obtained from the imaging sequence. The MR image may be corrected so that the centre of the MR image is aligned with the position of the MR active device.

The imaging sequence may be an MR thermometry sequence.

The controller may be configured to determine the position of the MR active device by processing the MR signals in the frequency domain so as to identify one or more signal peaks in the MR signals. The identified one or more signal peaks in the MR signals may correspond to the position of the MR active device in one or more spatial directions.

Prior to performing steps (a) to (e), the MR apparatus may be configured to perform a calibration phase to determine the predetermined trigger position. Performing the calibration phase may comprise: the controller controlling the transmitter and gradient arrangement to generate a plurality of the MR tracking sequences over time for tracking the position of the MR active device located in the subject; the controller obtaining MR signals detected by the MR active device as a result of the generated tracking sequences over time; the controller processing the obtained MR signals to determine how the position of the MR active device changes over time; and the controller using the information about how the position of the MR active device changes over time to set the predetermined trigger position.

It will be appreciated that any particular position of the MR active device may be set as the predetermined trigger position. In one example, the controller using the information about how the position of the MR active device changes over time to set the predetermined trigger position may comprise the controller setting an average position of the MR active device as the predetermined trigger position. That is, the controller may set an average position between a minimum and maximum position of the MR active device as the predetermined trigger position. In another example, the controller using the information about how the position of the MR active device changes over time to set the predetermined trigger position may comprises the controller setting a minimum or maximum position of the MR active device as the predetermined trigger position.

The MR apparatus may be configured to perform the method described above in relation to the first aspect of the disclosure.

### BRIEF DESCRIPTION OF THE DRAWINGS

Examples of the present disclosure will now be described with reference to the accompanying drawings, in which:

FIG. 1 is a process flow diagram for an example method according to a first aspect of the present disclosure;

FIG. 2 is an MR pulse sequence chart for an example method according to aspects of the present disclosure;

FIG. 3 is a timing diagram for an MR tracking sequence according to aspects of the present disclosure;

FIGS. 4A and 4B are MR images of a dynamic heart phantom in which a catheter with an embedded receive coil has been located;

FIGS. 5A to 5C are graphs showing the position of the receive coil over time and the time between detected triggers according to aspects of the present disclosure;

FIG. 6 is a series of MR thermometry images obtained using the approach of the present disclosure and using existing approaches;

FIG. 7 is a graph showing the myocardium area estimated from MR thermometry images obtained using the approach of the present disclosure and using existing approaches; and

FIG. 8 is a block diagram of an example MR apparatus and invasive device according to aspects of the present disclosure.

### DETAILED DESCRIPTION

The below examples all relate to tracking an MR active device in the form of a receive coil in a cardiac region of a subject. It will be appreciated that the present disclosure is not limited to this particular implementation and other forms of MR active device located within other regions of the subject can be tracked and used to trigger the generation of imaging sequences in accordance with the present disclosure.

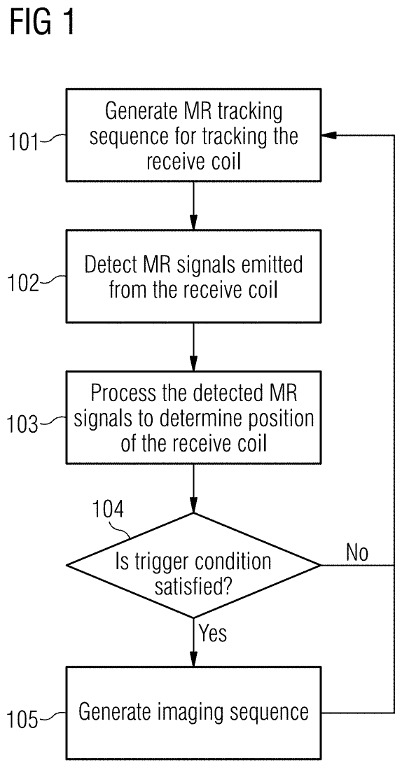

Referring to FIG. 1, there is shown a process flow diagram of an example method performed by an MR apparatus for triggering the generation of an imaging sequence for imaging a cardiac region of a subject according to aspects of the present disclosure.

Step 101 of the method comprises generating an MR tracking sequence for tracking the position of a receive coil located in a cardiac region of a subject. The receive coil is associated with an invasive device such as a catheter and is embedded in the catheter.

Step 102 of the method comprises obtaining MR signals detected by the receive coil as a result of the generated tracking sequence. It will be appreciated that the receive coil is communicatively coupled to the MR apparatus such that MR signals detected by the receive coil are provided to the MR apparatus.

Step 103 of the method comprises processing the obtained MR signals to determine the position of the receive coil.

Step 104 of the method comprises determining whether a trigger condition is satisfied by comparing the determined position of the receive coil to a predetermined trigger position.

If the trigger condition is satisfied, the method proceeds to step 105 which comprises generating the imaging sequence. The method then returns to step 101. If the trigger condition is not satisfied, the method returns to step 101, i.e. without triggering an imaging sequence.

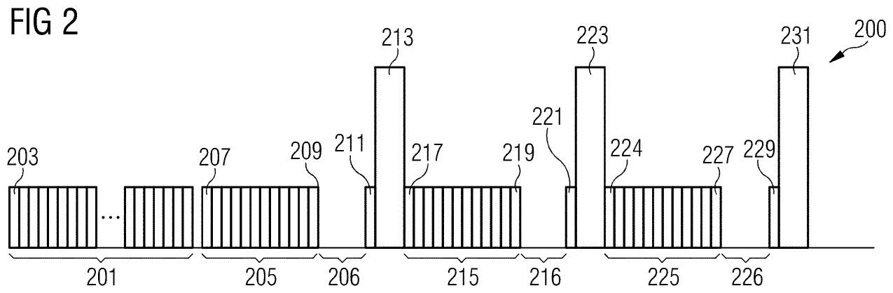

Referring to FIG. 2, there is shown a schematic diagram of a tracking and imaging sequence 200 according to the present disclosure.

The tracking and imaging sequence 200 comprises a calibration phase 201. The calibration phase 201 comprises generating a plurality of MR tracking sequences 203 in succession for tracking the position of the receive coil located in the cardiac region of the subject. The calibration phase 201 also comprises obtaining a plurality of MR signals detected by the receive coil as a result of the generated tracking sequences 203. The calibration phase 201 occurs for a period of time sufficient for the receive coil to be tracked over a number of cardiac cycles of the subject. In other words, the calibration phase 201 allows for the measurement of the receive coil over several cardiac cycles.

The calibration phase 201 further comprises processing the obtained MR signals to determine the position of the receive coil and how the position of the receive coil changes over the time during which the plurality of MR tracking sequences 203 are generated. In this particular example, the MR tracking sequences are for tracking the change in position of receive coil in 3D space. This information is represented as normalised 3D amplitude information which shows how the 3D amplitude of the receive coil changes over time relative to a reference position. In other examples, the position of the receive coil may be tracked in 2D or 1 D.

The calibration phase 201 further comprises using the information about how the position of the receive coil changes over time to set the predetermined trigger position. In one example, this comprises setting the maximum or minimum position of the receive coil determined from the calibration phase 201 as the predetermined trigger position. In another example, the average position representing the position of the receive coil between the maximum and minimum position of the receive coil is set as the predetermined position. Of course, these are just examples and any position of the receive coil can be selected as the predetermined trigger position. In particular, any position of the receive coil that corresponds to a position of the receive coil that occurs during the cardiac cycle can be used as a trigger position to trigger the generation of the imaging sequence.

Following the calibration phase 201, the tracking and imaging sequence 200 comprises a tracking phase 205. During the tracking phase, a plurality of tracking sequences 207 are generated until the trigger condition 209 is satisfied. This means that a tracking sequence 207 is generated, the MR signals detected by the receive coil as a result of the tracking sequence 207 are obtained, the obtained MR signals are processed to determine the position of the receive coil. It is then determined whether the trigger condition 209 is satisfied by comparing the determined positions of the receive coil to a predetermined trigger position. The trigger condition 209 may be satisfied if the determined position of the receive coil corresponds to the predetermined trigger position and/or if the position of the receive coil has exceeded the predetermined trigger position.

If the trigger condition 209 is not satisfied then the process repeats so that another tracking sequence 207 is generated, the MR signals detected by the receive coil as a result of the tracking sequence 207 are obtained, the obtained MR signals are processed to determine the position of the receive coil, and it is again determined whether the trigger condition is satisfied. In the example of FIG. 2, it can be seen that 12 tracking sequences 207 are generated in succession before the trigger condition 209 is satisfied.

Once the trigger condition 209 is satisfied, a predetermined time delay 206 is allowed to elapse before the imaging sequence 213 is generated for imaging the cardiac region of the subject. The predetermined time delay 206 is provided to ensure that the imaging sequence 213 is generated during a particular phase of the cardiac cycle of the subject. For example, so that the imaging sequence 213 is generated during the quiescent phase of the cardiac cycle. After the predetermined time delay 206 and prior to generating the imaging sequence 213, a further tracking sequence 211 is generated and the signals detected by the receive coil as a result of the further tracking sequence 211 are obtained. The obtained MR signals are used to determine the position of the receive coil just prior to the imaging sequence 213. This information is used to reposition the MR image obtained during the imaging sequence.

After the imaging sequence 213 is generated, another tracking phase 215 is performed which again comprises generating a plurality of tracking sequences 217 until the trigger condition 219 is satisfied. Once the trigger condition 219 is satisfied, a predetermined time delay 216 is provided before the imaging sequence 223 is generated. After the predetermined time delay 216 and prior to generating the imaging sequence 223, a further tracking sequence 221 is generated and the signals detected by the receive coil as a result of the further tracking sequence 221 are obtained. The obtained signals emitted from the receive coil are used to determine the position of the receive coil just prior to the imaging sequence 223. This information is used to reposition the MR image obtained during the imaging sequence.

After the imaging sequence 223 is generated, another tracking phase 225 is performed which again comprises generating a plurality of tracking sequences 224 until the trigger condition 227 is satisfied. Once the trigger condition 227 is satisfied, a predetermined time delay 226 is provided before the imaging sequence 231 is generated. After the predetermined time delay 226 and prior to generating the imaging sequence 231, a further tracking sequence 229 is generated and the signals detected by the receive coil as a result of the further tracking sequence 229 are obtained. The detected signals emitted from the receive coil are used to determine the position of the receive coil just prior to the imaging sequence 231. This information is used to reposition the MR image obtained during the imaging sequence.

The calibration phase 201 may last for approximately 10 seconds. Each MR tracking sequence 203, 207, 211, 217, 221, 224, 229 may last for approximately 25 milliseconds. The predetermined time delays 206, 216, 226 in this example are 500 milliseconds, but it will be appreciated that this time delay depends on the predetermined trigger position.

If will be appreciated that further tracking phases can be provided to trigger additional imaging sequences if desired.

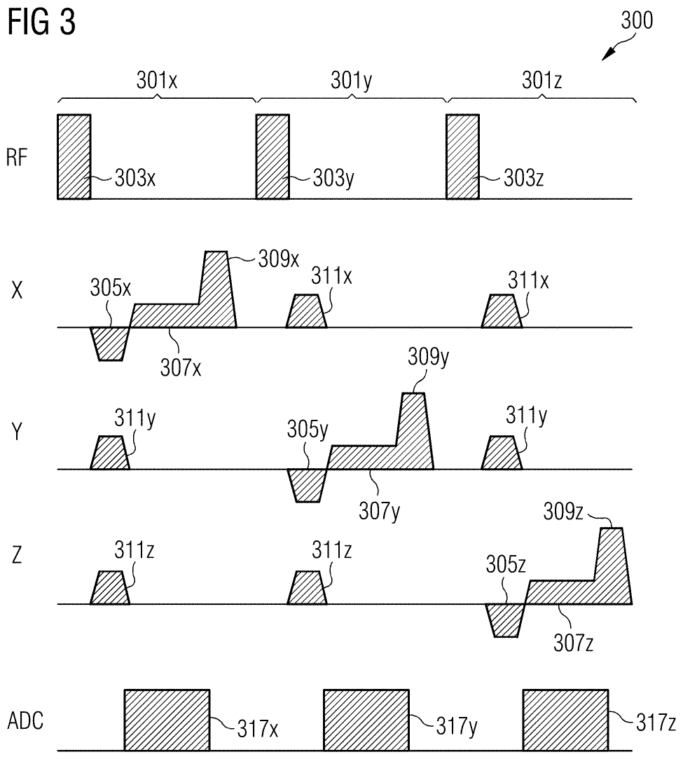

Referring to FIG. 3, there is shown a timing diagram for an example tracking sequence 300 according to the present disclosure which is useable in the calibration and tracking phases 201, 205, 215, 225 and tracking sequences 211, 221, 229 as described above in relation to FIG. 2. The timing diagram shows the relationship between excitation pulses, magnetic field gradients and data acquisitions during the tracking sequence 300.

This tracking sequence 300 is used to determine the position of the receive coil in 3D space and comprises three non-selective projection acquisitions 301x, 301y, 301z which are used to determine the position of the receive coil in a respective one of the x, y, and z directions. This is just one example tracking sequence for use in the present disclosure. The present disclosure is not limited to determining the position of the MR device in 3 dimensions. Instead, the position of the MR device may be determined in 2 dimensions by, for example, using just two of the non-selective projection acquisitions 301x, 301y, 301z shown in FIG. 3 or in 1 dimension by, for example, using just one of the non-selective projection acquisitions 301x, 301y, 301z shown in FIG. 3. That is, the position of the MR active device may be determined in 1 D, 2D, or 3D. Tracking fewer dimensions may save time as each tracking sequence 300 will be shorter, but the triggering of the MR imaging sequence may be less robust.

The first non-selective projection acquisition 301x is for detecting the position of the receive coil in the x direction. The first non-selective projection acquisition 301 comprises a first spatially non-selective excitation pulse 303x applied using an RF transmit coil of the MR apparatus. The first spatially non-selective excitation pulse 303x is applied to excite all spins within a large volume inside the RF transmit coil. In some examples, a weakly spatially selective RF excitation pulse 303 is used instead of a spatially non-selective excitation pulse.

Shortly after the first spatially non-selective excitation pulse 303x, a first magnetic field gradient pulse 305x is applied along the x direction. At the same time, a second magnetic field gradient pulse 311y is generated along the y direction and a third magnetic field gradient pulse 311z is generated along the z direction. The second and third magnetic field gradient pulses 311y, 311z have the opposite polarity to the first magnetic field gradient pulse 305x. The first, second and third magnetic field gradients 305x, 311y, 311z have a dephasing effect and act to shift the centre of the MR echo, e.g. to be outside of the period when the data acquisition signal 317x is generated. This advantageously is able to suppress broad features in the data while retaining narrow features such as the signal detected by the receive coil.

Following the first, second and third magnetic field gradient pulses 305x, 311y, 311z a fourth magnetic field gradient pulse 307x is applied along the x-direction having the opposite polarity to the first magnetic field gradient pulse 305x. The fourth magnetic field gradient pulse 307x in this example has twice the amplitude of the first magnetic field gradient pulse 305x. The fourth magnetic field gradient pulse 307x has the effect of making the magnetic field vary monotonically with the position along the x direction. This means that the frequency of the spins at different locations, which is also the frequency of the received MR signal, linearly depends on the spins' locations. The receive coil has a limited receive sensitivity profile which means that it can only detect spins in the immediate vicinity of the receive coil. As a consequence, the MR signal received by the receive coil is shown as a sharp peak in the frequency spectrum. The location of the signal peak in the frequency domain indicates the spatial location of the receive coil along the axis of the applied gradient, i.e. the x-direction. The combination of the first magnetic field gradient pulse 305x and fourth magnetic field gradient pulse 307x results in the generation of a gradient echo which creates a first MR signal. During the fourth magnetic field gradient pulse 307x, the data acquisition signal 317x is generated to cause the first MR signal to be received by the receive coil and consequently the MR apparatus. The location of the signal peak of the MR signal in the frequency domain indicates the spatial location of the receive coil in the x-direction.

Following the fourth magnetic field gradient pulse 307x, a fifth magnetic field gradient pulse 309x is generated along the x-direction and having the same polarity as the fourth magnetic field gradient pulse 307x. The fifth magnetic field gradient pulse 309x is a spoiler pulse that is intended to ensure that before the generation of the second non-selective excitation pulse 303y, the steady-state magnetization does not have transverse components.

The second non-selective projection acquisition 301y is similar to the first non-selective acquisition 301x but is applied to determine the position of the receive coil in the y direction rather than the x direction.

The second non-selective projection acquisition 301y comprises a second spatially non-selective RF pulse 303y Shortly after the RF excitation pulse 303y, a first magnetic field gradient pulse 305y is applied along the y direction, a second magnetic field gradient pulse 311x is generated along the x direction and a third magnetic field gradient pulse 311z is generated along the z direction. The second and third magnetic field gradient pulses 311x, 311z have the opposite polarity to the first magnetic field gradient pulse 305y.

Following the first, second and third magnetic field gradient pulses 305y, 311x, 311z a fourth magnetic field gradient pulse 307y is applied along the y-direction having the opposite polarity to the first magnetic field gradient pulse 305y and having twice the amplitude of the first magnetic field gradient pulse 305y. During the fourth magnetic field gradient pulse 307y, the data acquisition signal 317y is generated to cause a second MR signal created as a result of a gradient echo generated using the combination of the first magnetic field gradient pulse 305y and the fourth magnetic field gradient pulse 307y to be received by the receive coil and consequently the MR apparatus. The location of the signal peak of the second MR signal in the frequency domain indicates the spatial location of the receive coil in the y-direction.

Following the fourth magnetic field gradient pulse 307y, a fifth magnetic field gradient pulse 309y is generated along the y-direction and having the same polarity as the fourth magnetic field gradient pulse 307y. The fifth magnetic field gradient 309y is a spoiler pulse that is intended to ensure that before the generation of the third non-selective excitation pulse 303z, the steady-state magnetization does not have transverse components.

The third non-selective projection acquisition 301z is similar to the first and second non-selective acquisitions 301x, 301y but is applied to determine the position of the receive coil in the z direction rather than the x and y directions.

The third non-selective projection acquisition 301z comprises a second spatially non-selective RF pulse 303z. Shortly after the RF excitation pulse 303z, a first magnetic field gradient 305z is applied along the z direction, a second magnetic field gradient pulse 311x is generated along the x direction and a third magnetic field gradient pulse 311y is generated along the y direction. The second and third magnetic field gradient pulses 311x, 311y have the opposite polarity to the first magnetic field gradient pulse 305z.

Following the first, second and third magnetic field gradient pulses 305z, 311x, 311y a fourth magnetic field gradient pulse 307z is applied along the z-direction having the opposite polarity to the first magnetic field gradient pulse 305z and having twice the amplitude of the first magnetic field gradient pulse 305z. During the fourth magnetic field gradient pulse 307z, the data acquisition signal 317z is generated to cause a third MR signal created as a result of a gradient echo generated using the combination of the first magnetic field gradient pulse 305z and the fourth magnetic field gradient pulse 307z to be received by the receive coil and consequently the MR apparatus. The location of the signal peak of the third MR signal in the frequency domain indicates the spatial location of the receive coil in the z-direction.

Following the fourth magnetic field gradient pulse 307z, a fifth magnetic field gradient pulse 309z is generated along the z-direction and having the same polarity as the fourth magnetic field gradient pulse 307z. The fifth magnetic field gradient pulse 309z is a spoiler pulse that is intended to ensure that before the generation of next tracking sequence, the steady-state magnetization does not have transverse components.

It will be appreciated that the tracking sequence 300 described in FIG. 3 results in first, second, and third MR signals which each contain information about the position of the receive coil in a respective one of the x, y and z directions. The frequency components of the MR signals contain a single peak which corresponds to the position of the receive coil in the respective one of the x, y and z directions. By extracting the location of the maximum value from each of the MR signals, the position of the receive coil in 3D space is able to be determined.

It will be appreciated that the de-phasing and spoiler gradients 305x,y,z, 309x,y,z and 311x,y,z are optional and not required in all arrangements of the present disclosure.

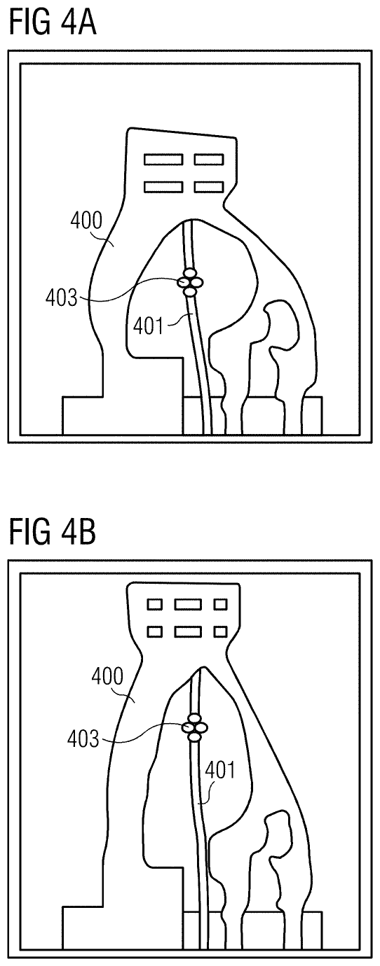

Referring to FIGS. 4A and 4B, there are shown MR localizer images of a dynamic heart phantom 400 during an example procedure. A dynamic heart phantom 400 is a model of the heart which can be driven move in a regular, repeatable way, and at a rate which can be selected to correspond to a human heartbeat. FIGS. 4A and 4B show that an invasive device in the form of a catheter 401 is positioned within the dynamic heart phantom 400. The catheter 401 comprises a receive coil (not shown) in the form of a microcoil embedded in the catheter 401. The catheter 401 has been maneuvered into a fixed position which mimics how a surgeon would manoeuvre a catheter during a cardiac ablation procedure. The catheter 401 moves due to the cardiac motion of the dynamic heart phantom 400.

FIG. 4A shows the dynamic heart phantom 400 in a compressed state which corresponds to the systolic period of the cardiac cycle. FIG. 4B shows the dynamic heart phantom 400 in a stretched state which corresponds to the diastolic period of the cardiac cycle. The motion of the dynamic heart phantom 400 during the cardiac cycle causes movement of the catheter 401 and the receive coil attached to the catheter. This motion can be viewed from FIG. 4A and FIG. 4B by comparing the relative position of the object 403 on the catheter 401 in each of these figures. The present disclosure tracks the motion of the receive coil using the MR tracking sequences described above to trigger the generating of an imaging sequence.

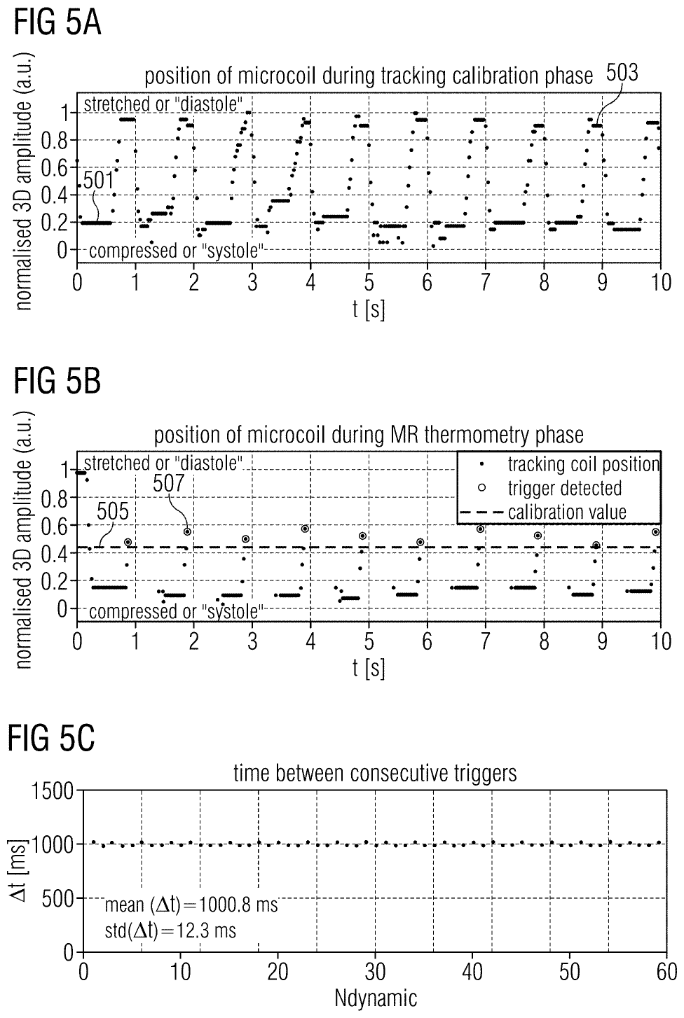

Referring to FIG. 5A there is shown a graph which compares the position of the receive coil of FIGS. 4A and 4B during the calibration phase 201 of the MR tracking and imaging sequence 200 shown in FIG. 2. The y axis of the graph represents the position of the receive coil, and in particular represents the position of the receive coil in terms of the normalised 3D amplitude. The x axis of the graph represents time in seconds.

It can be seen in FIG. 5A that the tracking sequences 203 generated during the calibration phase 201 enables the position of the receive coil in 3D space to be tracked. The receive coil moves in a way which mimics the cardiac motion of the heart. This is because the only motion of the receive coil is due to the cardiac motion of the heart during the cardiac cycle. The receive coil moves between a minimum amplitude position 501 that corresponds to the compressed or systolic period of the cardiac cycle and a maximum amplitude position 503 that corresponds to the stretched or diastolic period of the cardiac cycle. It will be appreciated that the minimum and maximum position 501, 503 may vary slightly across different cardiac cycles due to, for example, changes in the cardiac motion. The calibration phase 201 of this example takes the average position between the minimum and maximum positions 501, 503 over a multitude of cardiac cycles, as the predetermined trigger position.

Referring to FIG. 5B, there is shown a graph which compares the position of the receive coil of FIGS. 4A and 4B during the tracking phases of the tracking and imaging sequence 200 shown in FIG. 2. The y axis of the graph represents the position of the receive coil, and in particular represents the position of the receive coil in terms of the normalised 3D amplitude. The x axis of the graph represents time in seconds. The horizontal line 505 in FIG. 5B represents the predetermined trigger position 505. During each tracking phase 205, 215, 225, the position of the receive coil as detected by the tracking sequences 207, 209, 226 are compared to the predetermined trigger position 505. If the position 507 of the receive coil exceeds/surpasses the predetermined trigger position 505, and if the motion of the receive coil, determined by comparing the detected position of the receive coil with the previous position of the receive coil, has a positive slope (which indicates the receive coil is moving in one specific direction and excludes motion in the other direction), then the trigger condition is satisfied. It will be noted from FIG. 5B that after the trigger condition is satisfied, there is an absence of data related to the position of the receive coil. This is due to the predetermined trigger delay 206, 216, 226. In other examples, additional tracking sequences will be generated during the predetermined trigger delay 206, 216, 226.

Referring to FIG. 5C, there is shown a graph which compares the time between consecutive triggers for a number of performances of the tracking phase 205, 215, 225 on the dynamic heart phantom 400 and receive coil arrangement 403 shown in FIGS. 4A and 4B. The y axis of the graph represents the time between consecutive triggers in milliseconds. The x axis of the graph represents the number of trigger phases. In this experiment, the dynamic heart phantom 400 was driven at 60 beats per minute. The graph shows that the time between the consecutive triggers is substantially constant at 1000 ms across the trigger phases. The mean time is 1000.8 ms and the standard deviation is 12.3 ms. This corresponds to the driving rate of the dynamic heart phantom that was used in this experiment which was driven at 60 bpm.

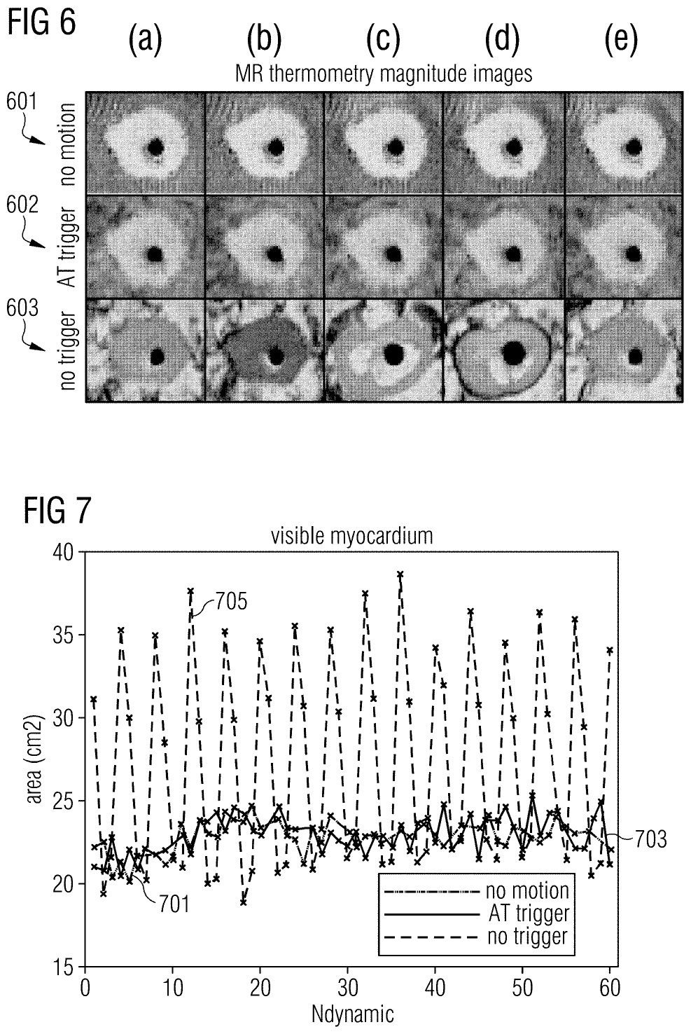

Referring to FIG. 6 there is shown a series of MR thermometry magnitude images of the cardiac region of the dynamic heart phantom 400 of FIGS. 4A and 4B. The images 601 (a) to (e) were obtained in the absence of cardiac motion. This means that there are no motion related artefacts in these images. The images 602 (a) to (e) were obtained when the dynamic heart phantom 400 was driven at 60 bpm. The method for triggering the imaging sequence as described above in relation to the present disclosure was used to trigger the MR thermometry imaging sequences. The images 603 (a) to (e) were obtained when the dynamic heart phantom 400 was driven at 60 BPM, but without the use of the triggering method of the present disclosure, instead the MR-controller was pre-programmed to generate imaging sequences at a rate of 80 BPM.

It can be appreciated from FIG. 6 that there is a high degree of correspondence between the images 601 (a) to (e) obtained without motion and the images 602 (a) to (e) obtained with motion but using the method of triggering the imaging sequences as provided by the present disclosure. In particular, the myocardial area in the images 601 (a) to (e) is similar to the myocardial area in the images 602 (a) to (e). By contrast, the images 603 (a) to (e) obtained without triggering generally do not correspond to the images 601 (a) to (e) and have a high degree of variation in myocardium area. This highlights the effectiveness of the present disclosure in triggering imaging sequences to as to avoid motion errors due to cardiac motion.

Referring to FIG. 7, there is shown a graph of the change in observed myocardial area across a number of generated images using the three imaging situations mentioned above in relation to FIG. 6. The y axis of the graph represents the observed area of the myocardium in the obtained thermometry image in cm squared. The x axis of the graph represents the number of images obtained. The plot 701 relates to the observed myocardium area in the absence of motion of the dynamic heart phantom 400. The plot 703 relates to the observed myocardium area when the dynamic heart phantom 400 is driven at 60 bpm and when the MR thermometry sequences are trigged by the method according to the present disclosure. The plot 705 relates to the observed myocardium area when the dynamic heart phantom 400 is driven at 60 bpm and when a triggering process is not used.

It can be seen that the plot 703 closely corresponds to the plot 701 which means that the area estimated in accordance with the present disclosure is consistent with the area estimated in the absence of cardiac motion. The plot 705 shows a large variation in the area size. The mean estimated area according to the plot 701 is 22.8 cm squared, and the standard deviation is 0.9 cm squared. The mean estimated area according to plot 703 is 22.9 cm squared and the standard deviation is 1.1 cm squared. The mean estimated area according to plot 705 is 27.2 cm squared and the standard deviation is 6.2 cm squared. This highlights the effectiveness of the present disclosure in triggering imaging sequences to as to avoid motion errors due to cardiac motion.

In an example use, a surgeon will navigate the catheter 401 (FIG. 4A) to a desired position for performing a cardiac ablation. During the process of performing the cardiac ablation, the catheter 401 remains in a fixed position and thus the only motion of the catheter 401 is due to cardiac motion. The method according to the present disclosure is performed at this time to trigger the generation of the imaging sequences, such as MR thermometry sequences. The surgeon may then move to a new location to perform another cardiac ablation. The method according to the present disclosure may then be repeated to trigger the generation of additional imaging sequences. The approaches of the present disclosure therefore generally require that the catheter 401/receive coil 403 (FIG. 4A) is held in a generally fixed position so that the only (or at least a major) component of the motion of the catheter 401/receive coil 403 is due to cardiac motion. Of course, the catheter 401/receive coil 403 can be moved between different performances of the method according to the present disclosure.

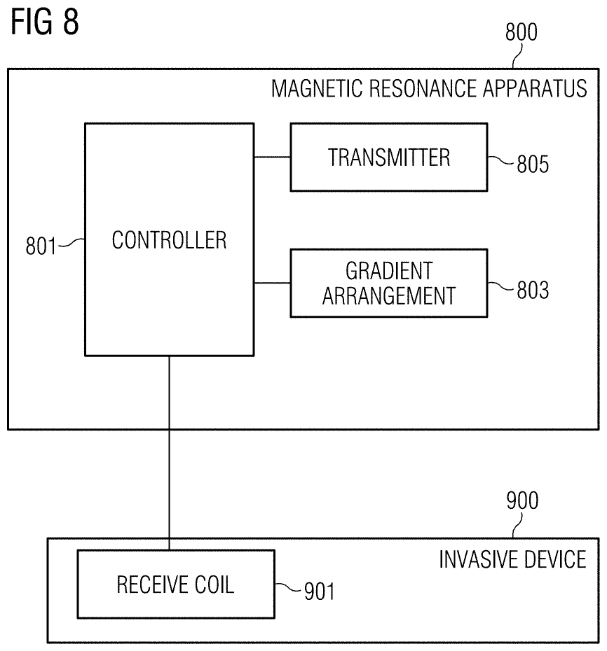

Referring to FIG. 8, there is shown a block diagram of an example MR apparatus 800 and invasive device 900 according to aspects of the present disclosure. The MR apparatus 800 and invasive device 900 may together form a system.

The MR apparatus 800 comprises a controller 801, gradient arrangement 803 and transmitter 805. The controller 801 is in communication with the transmitter 805, and the gradient arrangement 803 for controlling these components. The gradient arrangement 803 configured to apply a magnetic field gradient, and in particular a gradient arrangement configured to generate magnetic field gradients along three mutually orthogonal direction x, y, z. The MR apparatus 800 further comprises a transmitter 805 configured to apply an excitation pulse (e.g. an RF excitation pulse) to the subject positioned within the MR apparatus 800.

The controller 801 is communicatively coupled to a receive coil 901 associated with an invasive device 900 which is positioned in a subject that is being imaged by the MR apparatus 800. The receive coil 901 is positioned in the cardiac region of the subject. In this way, MR signals detected by the receive coil 901 are able to be provided to the controller.

The controller 801 is configured to control the transmitter 805 and gradient arrangement 803 to generate an MR tracking sequence for tracking the position of the receive coil 901. The controller 801 is configured to obtain MR signals detected by the receive coil 901 as a result of the generated tracking sequence. The controller 801 is configured to process the obtained MR signals to determine the position of the receive coil 901. The controller 801 is configured to determine whether a trigger condition is satisfied by comparing the determined position of the receive coil 901 to a predetermined trigger position. The controller 801 is further configured to control the transmitter 805 and gradient arrangement 803 to generate the imaging sequence if the trigger condition is satisfied. If the trigger condition is not satisfied, the imaging sequence is not generated.

The MR apparatus 800 includes a magnet (not shown) for establishing a stationary magnetic field. The magnet can include a permanent magnet, a superconducting magnet or other type of magnet. The transmitter 805 may be part of an excitation system (not shown) that may also comprise a receiver (not shown). The excitation system can be an RF system with one or more RF coils (not shown). The gradient arrangement comprises one or more coils (not shown) used to apply magnetic gradients for localization during MR imaging.

The controller 801 could be an integrated component of the MR apparatus 800. The controller 801 could be a desktop computer, a workstation, a server, or a laptop computer.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements.

The described and illustrated embodiments are to be considered as illustrative and not restrictive in character, it being understood that only the preferred embodiments have been shown and described and that all changes and modifications that come within the scope of the disclosures as defined in the claims are desired to be protected. It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the disclosure as defined in the appended claims. In relation to the claims, it is intended that when words such as “a,” “an,” “at least one,” or “at least one portion” are used to preface a feature there is no intention to limit the claim to only one such feature unless specifically stated to the contrary in the claim. When the language “at least a portion” and/or “a portion” is used the item can include a portion and/or the entire item unless specifically stated to the contrary.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The disclosure is not restricted to the details of the foregoing embodiment(s). The disclosure extends to any novel one, or any novel combination, of the features disclosed in this specification (including any accompanying claims, abstract and drawings), or to any novel one, or any novel combination, of the steps of any method or process so disclosed.

1. A method performed by a magnetic resonance (MR) apparatus for controlling a generation of an imaging sequence for imaging a subject, the method comprising:
(a) generating an MR tracking sequence for tracking a position of an MR active device located in the subject;
(b) obtaining MR signals detected by the MR active device as a result of the generated tracking sequence;
(c) processing the obtained MR signals to determine the position of the MR active device;
(d) determining whether a trigger condition is satisfied by comparing the determined position of the MR active device to a predetermined trigger position; and
(e) generating the imaging sequence if the trigger condition is satisfied, wherein if the trigger condition is not satisfied, the imaging sequence is not generated.

2. The method as claimed in claim 1, wherein if the trigger condition is not satisfied, the method further comprising repeating the steps (a) to (e).

3. The method as claimed in claim 2, wherein if the trigger condition is not satisfied, the method further comprising repeating the steps (a) to (e) until the trigger condition is satisfied.

4. The method as claimed in claim 1, wherein the generating the imaging sequence comprises generating the imaging sequence a predetermined time after the trigger condition is determined to be satisfied.

5. The method as claimed in claim 4, wherein the predetermined time is selected such that the imaging sequence is generated during a specified time point in a motion cycle of the subject such as a cardiac cycle or a respiratory cycle.

6. A method as claimed in claim 5, wherein the specified time point corresponds to a quiescent phase of the motion cycle.

7. The method as claimed in claim 1, wherein the steps (a) to (c) are performed immediately prior to the step (e).

8. The method as claimed in claim 7, the method further comprising:
using the determined position of the MR active device to correct the position of an MR image obtained from the imaging sequence, wherein the MR image is aligned with the position of the MR active device.

9. The method as claimed in claim 7, further comprising:
using the determined position of the MR active device to determine if the position of the MR active device immediately prior to the generation of the imaging sequence corresponds to a quiescent phase of a motion cycle of the subject,
wherein the imaging sequence is not generated and the steps (a) to (e) are repeated if it is determined that the position of the MR active device immediately prior to the generation of the imaging sequence does not correspond to the quiescent phase of the motion cycle.

10. The method as claimed in claim 1, wherein the imaging sequence is an MR thermometry sequence.

11. The method as claimed in claim 1, wherein the processing the obtained MR signals to determine the position of the MR active device comprises:
processing the MR signals in the frequency domain so as to identify one or more signal peaks in the MR signals,
wherein the identified one or more signal peaks in the MR signals correspond to the position of the MR active device in one or more spatial directions.

12. The method as claimed in claim 1, wherein prior to performing the steps (a) to (e), the method comprises performing a calibration phase to determine the predetermined trigger position, wherein performing the calibration phase comprises:
generating a plurality of the MR tracking sequences over time for tracking the position of the MR active device located in the subject;
obtaining MR signals detected by the MR active device as a result of the generated plurality of MR tracking sequences over time;
processing the obtained MR signals to determine how the position of the MR active device changes over time; and
using the information about how the position of the MR active device changes over time to set the predetermined trigger position.

13. The method as claimed in claim 12, wherein the using the information about how the position of the MR active device changes over time to set the predetermined trigger position comprises setting an average position of the MR active device as the predetermined trigger position.

14. The method as claimed in claim 12, wherein the using the information about how the position of the MR active device changes over time to set the predetermined trigger position comprises setting a minimum or maximum position of the MR active device as the predetermined trigger position.

15. The method as claimed in claim 1, wherein the MR tracking sequence comprises a spatially non-selective or minimally spatially selective excitation pulse followed by a magnetic field gradient pulse along a first spatial direction, and wherein obtaining the MR signals detected by the MR active device as a result of the generated tracking sequence comprises obtaining first MR signals detected as a result of the magnetic field gradient pulse along the first spatial direction, wherein the location, in the frequency domain, of the signal peak for the first MR signals corresponds to the position of the MR active device in the first spatial direction.

16. The method as claimed in claim 15,
wherein the MR tracking sequence further comprises a spatially non-selective or minimally spatially selective excitation pulse followed by a magnetic field gradient pulse along a second spatial direction perpendicular to the first spatial direction, and wherein obtaining the MR signals detected by the MR active device as a result of the generated tracking sequence further comprises obtaining second MR signals detected as a result of the magnetic field gradient pulse along the second spatial direction, wherein the location, in the frequency domain, of the signal peak for the second MR signals corresponds to the position of the MR active device in the second spatial direction, and
wherein the MR tracking sequence further comprises a spatially non-selective or minimally spatially selective excitation pulse followed by a magnetic field gradient pulse along a third spatial direction perpendicular to the first spatial direction and second spatial direction, and wherein obtaining the MR signals detected by the MR active device as a result of the generated tracking sequence further comprises obtaining third MR signals detected as a result of the magnetic field gradient pulse along the third spatial direction, wherein the location, in the frequency domain, of the signal peak for the third MR signals corresponds to the position of the MR active device in the third spatial direction.

17. The method as claimed in claim 1, wherein the determining whether the trigger condition is satisfied comprises determining whether the determined position of the MR active device corresponds to or exceeds the predetermined trigger position.

18. The method as claimed in claim 1, wherein the imaging sequence is for imaging a cardiac region of the subject, and wherein the step (a) comprises generating the MR tracking sequence for tracking the position of the MR active device located in the cardiac region of the subject.

19. The method as claimed in claim 1, wherein the imaging sequence is for imaging an abdomen, kidney, or pancreas of the subject, and wherein the step (a) comprises generating the MR tracking sequence for tracking the position of the MR active device located in the abdomen, kidney, or pancreas of the subject

20. The method as claimed in claim 1, wherein the MR active device is a receive coil.

21. The method as claimed in claim 1, wherein the MR active device is part of a catheter.

22. A magnetic resonance (MR) apparatus, comprising:
a gradient arrangement configured to apply a magnetic field gradient;
a transmitter configured to apply an excitation pulse to a subject; and
a controller configured to communicate with the transmitter and with the gradient arrangement for controlling these components,
wherein the controller is configured to:
(a) control the transmitter and gradient arrangement to generate an MR tracking sequence for tracking the position of an MR active device located in the subject;
(b) obtain MR signals detected by the MR active device as a result of the generated tracking sequence;
(c) process the obtained MR signals to determine the position of the MR active device;
(d) determine whether a trigger condition is satisfied by comparing the determined position of the MR active device to a predetermined trigger position; and
(e) control the transmitter and gradient arrangement to generate an imaging sequence for imaging the subject if the trigger condition is satisfied,
wherein if the trigger condition is not satisfied, the imaging sequence is not generated.

23. The MR apparatus as claimed in claim 22, wherein if the trigger condition is not satisfied, the controller is configured to repeat the performance of the steps (a) to (e).

24. The MR apparatus as claimed in claim 23, wherein if the trigger condition is not satisfied, the controller is configured to repeat the performance of (a) to (e) until the trigger condition is satisfied.

25. The MR apparatus as claimed in claim 22, wherein the controller is configured to control the transmitter and gradient arrangement to generate the imaging sequence a predetermined time after the trigger condition is determined to be satisfied.
